(19) United States
(12) Patent Application Publication
(10) Pub. No.: US 2014/0360501 A1
(43) Pub. Date: Dec. 11, 2014

(54) GAS PRESSURE MEASUREMENT SYSTEM FOR PATIENT VENTILATION APPARATUS

(71) Applicant: Air Liquide Medical Systems, Antony (FR)

(72) Inventors: Hadrien GUIDUCCI, Paris (FR); Damien Germani, Paris (FR)

(21) Appl. No.: 14/297,795

(22) Filed: Jun. 6, 2014

(30) Foreign Application Priority Data
Jun. 6, 2013 (FR) 1355204

(51) Int. Cl. G01L 7/02; A61M 16/08; G01L 7/08; A61M 16/00

(52) U.S. Cl. CPC G01L 7/022; A61M 16/0003; A61M 16/0057; A61M 16/0883; G01L 7/08; A61M 2016/0027
USPC 128/205.23; 73/715; 73/716

(57) ABSTRACT

The invention relates to a gas pressure measurement system comprising at least one gas circuit and at least one pressure sensor (6) arranged so as to be able to measure the pressure of the gas in at least part of the gas circuit, said at least one pressure sensor being protected by a protective membrane permeable to gas, arranged between said at least one pressure sensor and the gas circuit. It further comprises a flexible intermediate piece formed by an elastically deformable material arranged between said at least one pressure sensor and the protective membrane, said flexible intermediate piece comprising an internal passage putting the protective membrane and said at least one pressure sensor in fluid communication. Patient ventilation apparatus comprising a gas circuit able to convey gas between a gas source and a patient, as well as such a gas pressure measurement system.

### CROSS REFERENCE TO RELATED APPLICATIONS

This application claims the benefit of priority under 35 U.S.C. §119 (a) and (b) to French Patent Application No. 1355204, filed Jun. 6, 2013, the entire contents of which are incorporated herein by reference.

### BACKGROUND

The invention concerns a gas pressure measurement system comprising at least one pressure sensor and a protective membrane, in which a flexible intermediate piece ensures the gastightness of the sensor and of its pressure tapping while protecting them from moisture, in particular the moisture coming from the patient gas circuit of a medical ventilation apparatus of a patient comprising a ventilated gas circuit equipped with such a system.

In some medical appliances for measuring gas flow rate, the air going to the patient is humid. However, pressure sensors are very sensitive to humidity.

Moreover, the sensors have a variable geometry, that is to say with shapes and dimensions that may be substantially different from one sensor version to another.

Consequently the problem that is posed is being able to achieve gastightness of the pressure sensor of a patient circuit and to obtain protection thereof against moisture, whatever the sensor used, that is to say whatever its geometry.

In other words, there exists a requirement for a system making it possible to bring gas, such as air, or a gas pressure to the sensor while preventing leakage to the outside and moreover able to protect the sensor from humidity, or even to provide an antibacterial barrier.

Among the existing solutions, some systems are known using several rigid machined or moulded pieces associated with flexible gaskets and/or glue for holding the membrane protecting the sensor.

However, these solutions give rise to drawbacks or lead to other problems such as difficulties in assembly, impossibility of disassembling in the case where glue is used, a risk of forgetting gaskets during assembly, high costs relating to the number of parts and manipulations thereof, a risk of damage to the membrane, which is sometimes very fragile, by one or other of the rigid pieces used, then giving rise to unexpected leakages, a risk of damage to the gasket during assembly, a weak or even insufficient gastight zone, etc.

Other solutions including sensors having their own connection exist.

In this case, it is necessary then to connect the sensors and to the measuring orifices with connection means, such as pipes, connection bodies, etc.

However, these types of sensor are much more imposing. Thus the size thereof may be 10 to 20 times greater than that of conventional sensors, for example around 1 mm for a conventional sensor as against 10 to 20 mm for sensors with inherent connectivity.

This type of sensor therefore poses problems of insertion and of use of this type of sensor in some medical apparatus, in particular of reduced size, and therefore gives rise to an appreciable increase in the size thereof.

In other words, the existing gas pressure measurement systems are not completely without posing certain problems and giving rise to certain drawbacks.

Consequently the stated problem is to propose a system for measuring gas pressure for a medical apparatus that uses a reduced number of parts, is of low cost, presents less risk of leakage, is easy and rapid to assemble, leads to fewer risks of forgetting during assembly, can be dismantled in production and maintenance, gives rise to fewer risks of leakage with damage to gaskets and/or of damaging the fragile membrane or membranes, etc.

### SUMMARY

The solution is then a gas pressure measurement system comprising at least one gas circuit and at least one pressure sensor arranged so as to be able to measure the pressure of the gas in at least part of the gas circuit, said at least one pressure sensor being protected by a protective membrane permeable to gas, arranged between said at least one pressure sensor and the gas circuit, characterised in that it further comprises a flexible intermediate piece formed by an elastically deformable material arranged between said at least one pressure sensor and the protective membrane, said flexible intermediate piece comprising an internal passage putting the protective membrane and said at least one pressure sensor in fluid communication.

According to circumstances, the gas pressure measurement system of the invention may comprise one or more of the following technical features:
it comprises two pressure sensors, each pressure sensor being protected by a protective membrane permeable to gas and a flexible intermediate piece formed by an elastically deformable material being arranged between each pressure sensor and the protective membrane;
it comprises two pressure sensors the pressure tappings of which are connected to the gas circuit on either side of a means for creating a pressure drop in said gas circuit, in particular a passage restriction;
the flexible intermediate piece has a shape of revolution;
the flexible intermediate piece comprising an internal passage that may be central or offset;
the flexible intermediate piece comprises an upstream internal recess situated on the same side as the protective membrane, and a downstream internal housing situated on the same side as said at least one pressure sensor, and the upstream internal recess and the downstream internal housing being fluidically connected to each other by the internal passage;
the upstream internal recess and the downstream internal housing have a gas passage diameter (or dimension) greater than that of the internal passage;
the flexible intermediate piece comprises a downstream internal housing wherein said at least one pressure sensor is positioned;
alternatively, the flexible intermediate piece comprises a downstream internal housing emerging around the pressure tapping orifice carried by said at least one pressure sensor;
the flexible intermediate piece is formed by a polymer or elastomer material, preferably a material of the thermoplastic elastomer TPE or silicone type;
the flexible intermediate piece comprises a downstream rim delimiting the downstream internal housing, said downstream rim bearing sealingly, around said at least one pressure sensor, on the wall carrying said at least one pressure sensor or on said at least one sensor itself, preferably around the pressure tapping orifice of said sensor or sensors;
the flexible intermediate piece comprises an upstream border delimiting the upstream internal recess and coming to bear on the protective membrane;
a casing (7) comprising a gas inlet orifice (7a) situated facing the upstream recess (8b), holds the flexible intermediate piece (8) in contact with the protective membrane (1) and in position around the pressure sensor (6);
the casing comprises a cover;
the casing comprises a first connecting piece carrying a gas inlet orifice and a second connecting piece carrying a gas outlet orifice, said gas inlet orifice and gas outlet orifice being connected by an internal gas passage, preferably said internal gas passage forms a part of the gas circuit;
the flexible intermediate piece comprises an external peripheral wall cooperating with the internal wall of the casing so as to provide a fluid seal between them;
the pressure sensor is carried by an electronic card forming all or part of the wall on which the flexible intermediate piece bears sealingly, via its downstream rim, around said pressure sensor;
the electronic card carrying the pressure sensor or sensors is fixed in the casing, preferably by screwing, so as to keep the protective membrane and the flexible intermediate piece integral with each other and also to provide a seal between the flexible intermediate piece and the wall carrying the pressure sensor;
the flexible intermediate piece comprises an external peripheral wall provided with outward wall expansions, that is to say having a form obtained by revolution, such as a lip or the like.

The invention further concerns a patient ventilation apparatus comprising a gas circuit able to convey a gas between a gas source and a patient, characterised in that it further comprises a gas pressure measurement system according to the invention, in particular as described above.

Preferably the gas pressure measurement system is arranged on the gas circuit, preferably in a casing connected fluidically to said gas circuit or to a bypass line of said gas circuit.

### BRIEF DESCRIPTION OF THE DRAWINGS

The invention will now be better understood by means of the following detailed description given with reference to the accompanying figures, among which:

FIG. 1 shows a first embodiment of the pressure measurement system according to the invention for an artificial ventilation apparatus,

FIG. 2 shows a view of a first side (the bottom face) of the flexible intermediate piece of the pressure measurement system of FIG. 1,

FIG. 3 shows a view of a second side (the top face) opposite to the first side of the flexible intermediate piece of the pressure measurement system of FIG. 1,

FIG. 4 is a schematic view of an embodiment of a patient ventilation apparatus equipped with a pressure measurement system according to FIG. 1 integrated in a casing, and

FIG. 5 is a diagram of a particular embodiment of the external peripheral wall of the flexible intermediate piece of FIGS. 1 to 3;

FIGS. 6A and 6B show an embodiment of the measurement system of FIG. 1 integrated in a casing connected to the gas circuit of an apparatus as illustrated in FIG. 4, and

FIG. 7 shows a second embodiment of the flexible piece of a pressure measurement system according to the invention.

### DESCRIPTION OF PREFERRED EMBODIMENTS

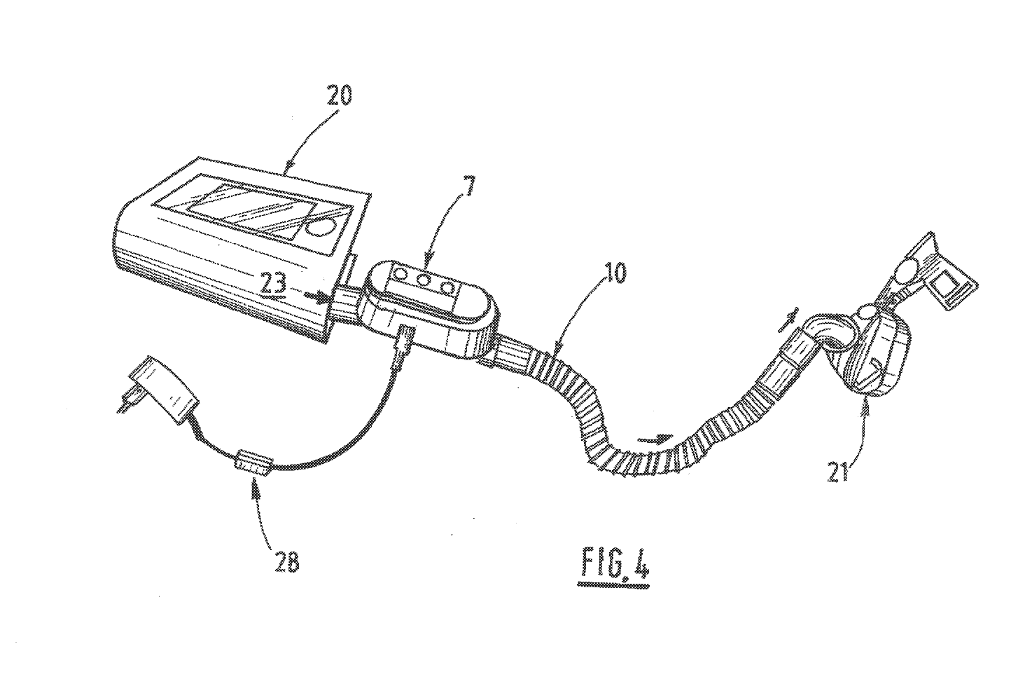

FIG. 4 shows a diagram of an embodiment of an artificial ventilation apparatus 20, that is to say a medical ventilator, such as for example the MONNAL™ T50 medical ventilator sold by the applicant, comprising a gas circuit 10, referred to as the patient circuit, which comprises a single respiratory branch, also referred to the inspiratory branch, fluidically supplied with gas by a gas source 23 (not visible), such as turbine or a microblower, situated in the apparatus 20, and which is able and designed to deliver a flow of gas under pressure, such as air, that is to say at a pressure above 1 atm, i.e. atmospheric pressure. The direction of the gas flow is shown schematically by the arrows along the inspiratory branch.

The patient circuit 10 conveys the gas issuing from the gas source 23 to a patient interface 21, such as a mask, which delivers the gas flow to a patient (not shown).

In order to be able to determine the pressure of the gas in at least part of the patient circuit 10, a gas pressure measurement system, 6, 16 is conventionally used, comprising one or more pressure sensors 6 and at least one electronic card 16 connected to said pressure sensor or sensors 6.

The pressure tapping or tappings 24 of said pressure sensor or sensors 6 communicate fluidically with the patient circuit 10 via one or more orifices 7a.

It is possible for example to use two pressure sensors 6 arranged in series with the membranes 1 and the deformable pieces 8 arranged in dedicated housings 34, 36.

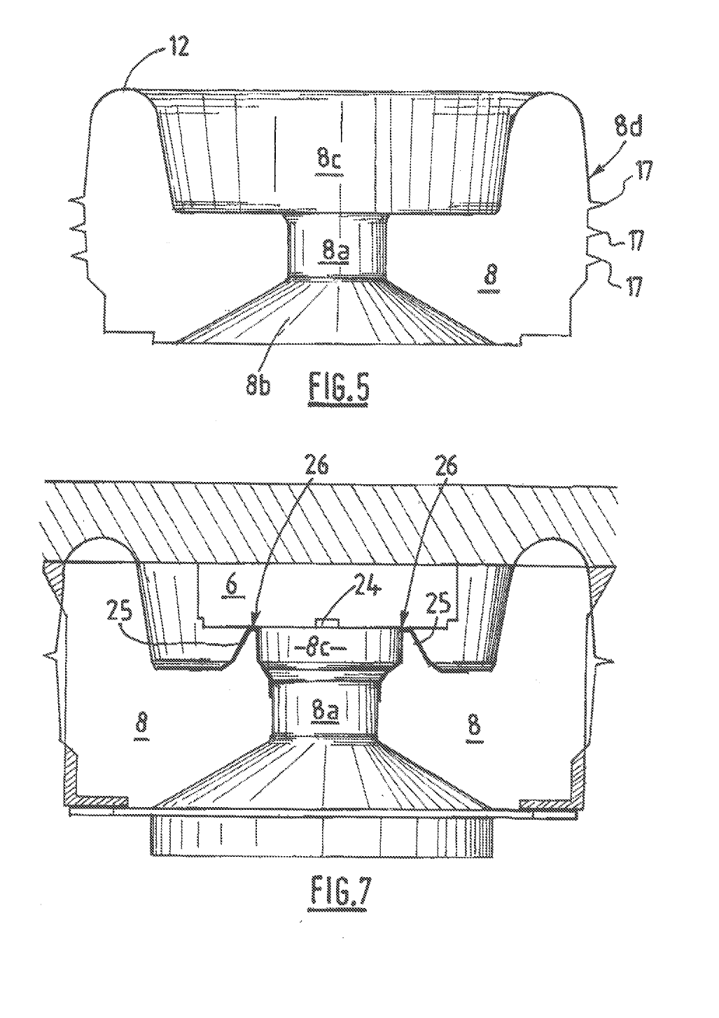

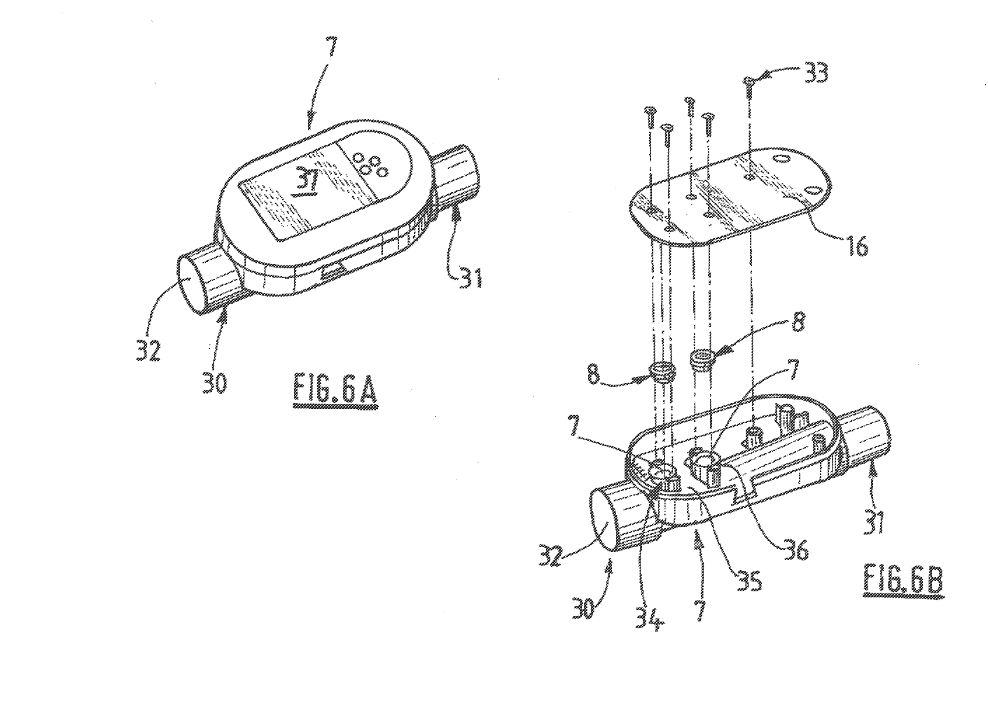

The sensors 6 have their pressure tappings 24 separated from each other by a means for creating pressure drops, such as a passage restriction 35 for example. Such an arrangement makes it possible in particular to make differential pressure measurements. Such an architecture is illustrated in FIG. 6B, as explained below.

In order to simplify understanding, the embodiment described below comprises only one pressure sensor 6 but the invention also aims to apply to pressure measurement systems with several sensors 6, in particular with two sensors.

Depending on the embodiment chosen, the pressure sensor or sensors 6 may be arranged so as to be able to measure the pressure of the gas flowing in the gas circuit 10:
either in the gas flow, that is to say as in FIG. 4, being integrated in a casing 7 connected the patient circuit 10, at the output of the ventilator 20 for example, and supplied with electric current by a conventional supply lead 28,
or in the wall of the circuit 10, that is to say being integrated in said wall for example,
or in a bypass line bringing the pressure or a flow of fluid to the sensor 6.

In order to protect the or each pressure sensor 6 from contaminants, such as water vapour (moisture), dust, microorganisms etc., liable to be found in the gas flow and/or in the patient circuit 10, the or each sensor 6 is protected by a protective membrane 1 permeable to the gas, arranged upstream of said sensor 6, in a housing or a bypass line making it possible to take a pressure tapping between the gas circuit 10 and the pressure sensor 6.

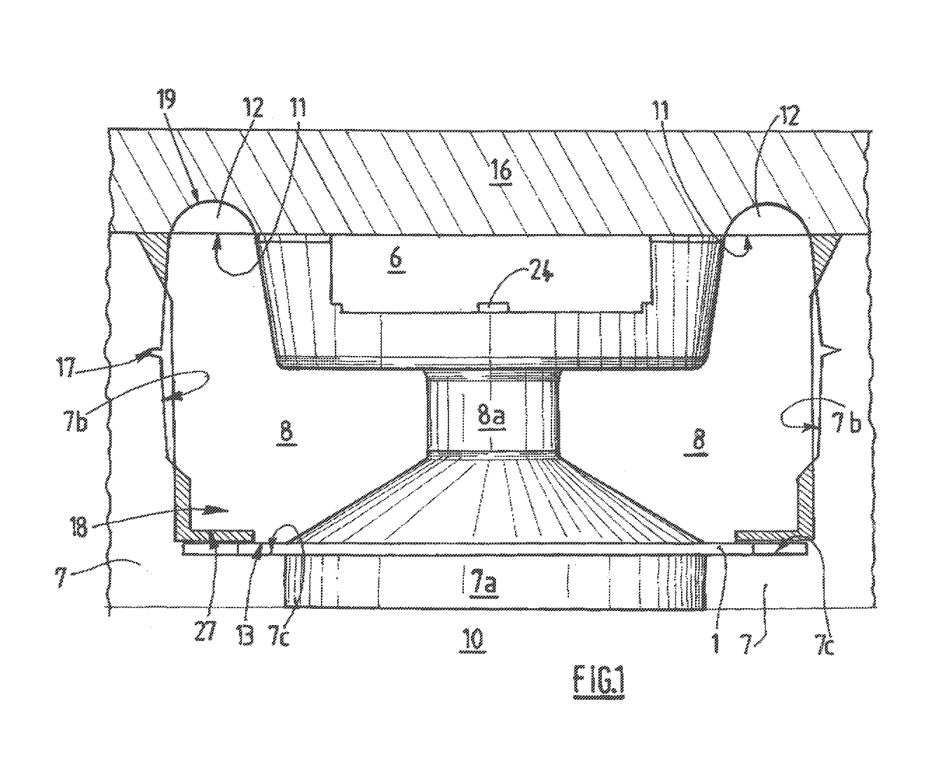

According to the present invention, as illustrated in FIG. 1, the pressure measurement system according to the invention further comprises a flexible intermediate piece 8 formed from an elastically deformable material arranged between the pressure sensor 6 and the protective membrane 1.

This flexible intermediate piece 8 comprises an internal passage 8a, such as a central passage, putting the protective membrane 1 and the pressure sensor 6 in fluidic communication so as to enable the gas pressure prevailing in the patient circuit 10 to be exerted successively through the membrane 1, in the internal passage 8a and as far as the pressure sensor 6, where the latter can measure it, via the pressure tapping 24 of the sensor 6, which is situated on or in the sensor 6. Here the pressure tapping 24 is an orifice enabling the pressure to enter inside the sensor 6.

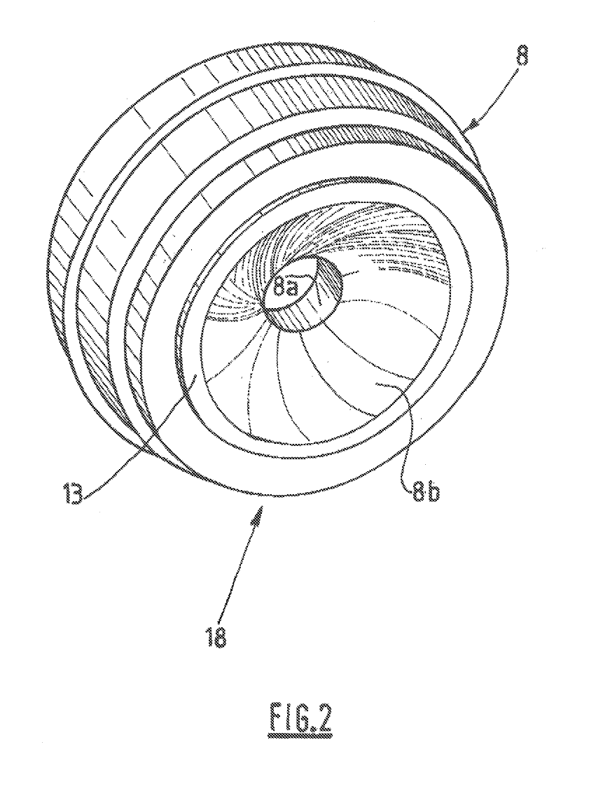

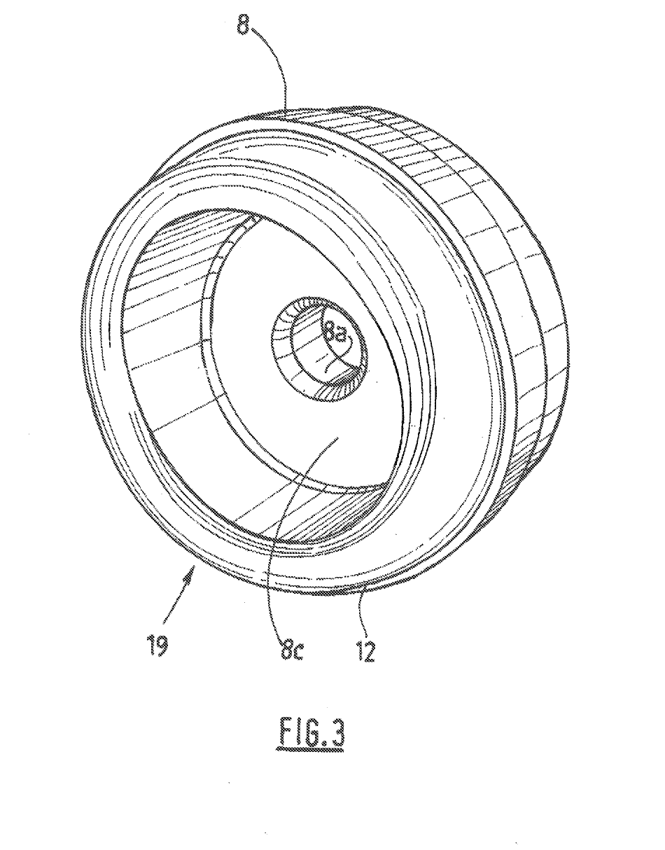

As can be seen in FIGS. 2 and 3, the flexible intermediate piece 8 is a piece of revolution, and therefore with a three-dimensional form overall. Here it comprises a circular periphery but this piece 8 may have other shapes, for example polygonal, in particular cubic, hexagonal or octagonal.

It further comprises an upstream internal recess 8b situated on the same side as the protective membrane 1, and a downstream internal housing 8c situated on the same side as the pressure sensor 6, which are fluidically connected to each other by the internal passage 8a.

Here the upstream internal recess 8b has a frustoconical form, that is to say the inside diameter thereof decreases progressively in the direction of the internal passage 8a.

However, the upstream internal recess 8b may have any other form provided that the fluid is brought to the sensor 6 through the passage 8a, which for its part forms a throttling neck, and the downstream internal housing 8c.

Moreover, concerning the downstream internal housing 8c, it should be emphasised that the sealing may also be done directly on the sensor 6 by modifying the form of said downstream internal housing 8c so as to reduce the volume of this downstream internal housing 8c so that it would form a chamber or a passage in line with the internal passage 8a, a wall expansion 25 of which would project rearwards and would come to bear directly (at 26) around the pressure tapping orifice 24 of the sensor 6 and create therein a gas seal, as illustrated by the particular embodiment in FIG. 7.

In the embodiment in FIG. 1, the flexible intermediate piece 8 comes to be positioned around the pressure sensor 6 so that its downstream internal housing 8c forms a protective chamber encompassing said pressure sensor 6.

In order to fulfill its function in particular of fluidic seal, the flexible intermediate piece 8 is formed from a polymer or elastomer material, preferably a material of the thermoplastic elastomer type, normally referred to as TPE, or silicone.

In fact, in the embodiment illustrated in FIGS. 1 and 3, the flexible intermediate piece 8 comprises, on the same side as its rear face 19, a downstream rim 12 delimiting the internal downstream housing 8c and bearing sealingly around the pressure sensor 6, that is to say on the wall 11 carrying the pressure sensor 6, in particular on an electronic card 16 that may constitute this wall 11 in certain embodiments.

This ensures a fluidic seal between the flexible intermediate piece 8 and the wall 11 carrying the pressure sensor 6, given that the elastic piece 8 deforms slightly in coming to be crushed against the wall 11 (FIG. 1) or against the sensor 6 itself (FIG. 7), depending on the embodiment considered.

As shown in FIG. 2, on its opposite face, that is to say the front face 18, the flexible intermediate piece 8 carries an upstream border 13 delimiting the upstream recess 8b and bearing on the protective membrane 1 and more precisely on the periphery of the rear face of the membrane 1 so as to hold it in position against the bottom 7c of the casing 7, as explained below in relation to FIG. 6B in particular.

This also provides a fluidic seal between the flexible intermediate piece 8 and the membrane 1. In particular, as illustrated in FIG. 1, a bearing 27 is provided here, in particular between the piece 8 and the membrane 1, in order to create a kind of gripping of the membrane 1; however, another shape could have been provided, such as a flat surface or a small lip of revolution.

In fact, the fluidic seal on the measuring system is provided by compression and deformation of the material of the flexible intermediate piece 8 that is sandwiched and slightly crushed and elastically deformed between the bottom 7c of the casing 7 and the wall 11 carrying the pressure sensor 6, as can be seen in FIG. 1.

This deformation and any shapes of revolution such as the fins 17 shown schematically in FIG. 5 provide the fluidic seal between the external peripheral wall 8d of the piece 8 and the internal surface 7c of the casing 7, in particular the internal surface 7c of one or more dedicated housings 34, 36 provided in the casing 7 and intended to receive the membrane or membranes 1 and the deformable piece or pieces 8, as illustrated in FIG. 6B.

This is because, in a particular embodiment, the flexible intermediate piece 8 comprises an external peripheral wall 8d provided with one or more wall expansions 17 projected towards the outside, preferably several wall expansions 17 of revolution arranged in parallel with one another, as shown schematically in FIG. 5. These wall expansions 17 improve the seal while reducing the radial crushing necessary.

Moreover, the wall 16 carrying the sensor or sensors 6, in particular an electronic card, is fixed by screwing so as to keep the protective membrane 1 and the flexible intermediate piece 8 secured to each other and also to provide a seal.

In fact, the flexible intermediate piece 8 falling within the scope of the present invention makes it possible to press the membrane 1 at the bottom 7c of the casing 7 and/or of a dedicated housing 34, 36, without damaging it, and thus provide a seal between the membrane 1 and the flexible intermediate piece 8 itself, while leaving the possibility for the fluid to pass through the membrane 1 and thus to be filtered.

On the opposite side, the flexible intermediate piece 8 provides a seal at the sensor 6, that is to say in the wall regions 11, 16 surrounding the sensor or sensors 6, that is to say the sensor 6 itself, the support wall or electronic card 16, etc.

Once fitted, the assembly also provides a seal with the outside and provides the pressure, i.e. the gas filtered by the membrane 1, such as air, to the or each pressure sensor 6.

Furthermore, the narrowing constituting all or part of the internal passage 8a of the flexible intermediate piece 8 also guarantees good rigidity of the assembly, which means that the whole of the flexible intermediate piece 8 forms a seal able to maintain an effective contact force on the sealing zones situated on the sides.

FIGS. 6A to 6B show an embodiment of the measurement system of FIG. 1 integrated in a casing 7 connected to the gas circuit 10 of a ventilator apparatus such as the one in FIG. 4. In FIG. 6A, the casing is seen closed, whereas FIG. 6B shows an exploded view of said casing 7.

More precisely, this casing 7 comprises an internal gas passage 32 forming a part of the gas circuit 10 connecting a gas inlet carried by an inlet connecting piece 30 to a gas outlet carried by an outlet connecting piece 31, between which the respiratory gas delivered by the gas source 23 of the ventilation apparatus 20 flows, supplying a patient via the interface 21 supplied by a flexible pipe forming another part of the gas circuit 10, as illustrated in FIG. 1.

In fact, the casing 7 is connected between the apparatus 20 and the flexible pipe 10 as illustrated in FIG. 1, at the inlet and outlet connecting pieces.

As can be seen in FIG. 6B, the casing 7 comprises here two pressure sensors (not visible) arranged in series and carried by the electronic card 16 on the same side as the face 11 for example in FIG. 6B, said electronic card 16 being fixed for example by screwing 33.

The deformable pieces 8 for their part are inserted in dedicated housings 34, 36.

Said sensors 6 moreover have their pressure tappings 24 spaced apart from each other while being separated by a pressure-drop creation means, for example a passage restriction 35 placed on the internal gas passage 32, which makes it possible to make differential pressure measurements.

The casing 7 is then closed by a cover 37, as illustrated in FIG. 6A, which protects the internal components and prevents entries of dust and other contaminants.

It should be emphasised that the pressure measurements made by the pressure sensor or sensors 6 are processed in a conventional manner by the electronic card 16, in particular by one or more microprocessors or the like.

In all cases, the advantages of a pressure measurement system of an apparatus according to the invention are in particular:
simplicity of assembly without an omission of a part being possible,
a very reduced assembly time,
reduced risk of leakage because of a smaller number of sealing zones, i.e. only three zones,
increased reliability since the membrane does not risk being damaged by a sharp piece etc.

The pressure measurement system and the ventilation apparatus equipped with such a pressure measurement system according to the invention can be used for supplying respiratory gas to patients suffering from respiratory problems, for example those suffering from respiratory pathologies for example of the ARDS or SAS (sleep apnoea) type and/or those that are to observe a treatment of the oxygen therapy or similar type.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A gas pressure measurement system (6, 16) comprising at least one gas circuit (10) and at least one pressure sensor (6) arranged so as to be able to measure the pressure of the gas in at least part of the gas circuit (10), said at least one pressure sensor (6) being protected by a protective membrane (1) permeable to gas, arranged between said at least one pressure sensor (6) and the gas circuit (10),
characterised in that the gas pressure measurement system (6, 16) further comprises a flexible intermediate piece (8) formed by an elastically deformable material arranged between said at least one pressure sensor (6) and the protective membrane (1), said flexible intermediate piece (8) comprising an internal passage (8a) putting the protective membrane (1) and said at least one pressure sensor (6) in fluid communication.

2. The gas pressure measurement system (6, 16) of claim 1, characterised in that the gas pressure measurement system (6, 16 comprises two pressure sensors (6), each pressure sensor (6) being protected by a protective membrane (1) permeable to gas and a flexible intermediate piece (8) formed by an elastically deformable material being arranged between each pressure sensor (6) and the protective membrane (1).

3. The gas pressure measurement system (6, 16) of claim 1, characterised in that the flexible intermediate piece (8) comprises an upstream internal recess (8b) situated on the same side as the protective membrane (1), and a downstream internal housing (8c) situated on the same side as said at least one pressure sensor (6), and the upstream internal recess (8b) and the downstream internal housing (8c) being fluidically connected to each other by the internal passage (8a).

4. The gas pressure measurement system (6, 16) of claim 1, characterised in that the flexible intermediate piece (8) comprises a downstream internal housing (8c) in which said at least one pressure sensor (6) is positioned.

5. The gas pressure measurement system (6, 16) of claim 1, characterised in that the flexible intermediate piece (8) is formed from a polymer or elastomer material.

6. The gas pressure measurement system (6, 16) of claim 1, characterised in that the flexible intermediate piece (8) comprises a downstream rim (12) delimiting the downstream internal housing (8c), said downstream rim (12) bearing sealingly, around said at least one pressure sensor (6), on a wall (11) carrying said at least one pressure sensor (6) or directly on said at least one sensor (6).

7. The gas pressure measurement system (6, 16) of claim 1, characterised in that the flexible intermediate piece (8) comprises an upstream border (13) delimiting the upstream internal recess (8b) and bearing on the protective membrane (1).

8. The gas pressure measurement system (6, 16) of claim 1, characterised in that a casing (7) comprising a gas inlet orifice (7a) situated opposite the upstream recess (8b) makes it possible to hold the flexible intermediate piece (8) in contact with the protective membrane (1) and in position around the pressure sensor (6).

9. The gas pressure measurement system (6, 16) of claim 1, characterised in that the flexible intermediate piece (8) comprises an external peripheral wall (8d) cooperating with the internal wall (7b) of the casing (7) so as to provide a fluidic seal between them.

10. The gas pressure measurement system (6, 16) of claim 6, characterised in that the pressure sensor (6) is carried by an electronic card (16) forming all or part of the wall (11) on which the flexible intermediate piece (8) bears sealingly, via its downstream rim (12), around said pressure sensor (6).

11. The gas pressure measurement system (6, 16) of claim 8, characterised in that an electronic card (16) carrying the sensor or sensors (6) is fixed in the casing (7) so as to keep the protective membrane (1) and the flexible intermediate piece (8) integral with each other and also to provide a seal between the flexible intermediate piece (8) and a wall (11) carrying the pressure sensor (6).

12. The gas pressure measurement system (6, 16) of claim 8, characterised in that the casing (7) comprises a first connecting piece (30) carrying a gas inlet orifice and a second connecting piece (31) carrying a gas outlet orifice, said gas inlet orifice and gas outlet orifice being connected by an internal gas passage (32), preferably said internal gas passage (32) forms part of the gas circuit (10).

13. A patient ventilation apparatus (20) comprising a gas circuit (10) able to convey gas between a gas source (23) and a patient, characterised in that the patient ventilation apparatus further comprises a gas pressure measurement system (6, 16) according to claim 1.

14. The patient ventilation apparatus according to claim 13, characterised in that the gas pressure measurement system (6, 16) is arranged on the gas circuit (10), in a casing (7) connected fluidically to said gas circuit (10) or to a bypass line of said gas circuit (10).
